(19) United States
(12) Patent Application Publication
(10) Pub. No.: US 2025/0073087 A1
(43) Pub. Date: Mar. 6, 2025

(54) NASAL LIQUID ABSORPTION DEVICE

(71) Applicant: Lucille Silva, Modesto, CA (US)

(72) Inventor: Lucille Silva, Modesto, CA (US)

(21) Appl. No.: 18/819,319

(22) Filed: Aug. 29, 2024

Related U.S. Application Data
(60) Provisional application No. 63/536,050, filed on Aug. 31, 2023.

(51) Int. Cl. A61F 13/12

(52) U.S. Cl. CPC A61F 13/126

(57) ABSTRACT

A nasal liquid absorption device to be disposed at least partially within a nose of a user, the nasal liquid absorption device including an attachment body to removably connect to the nose of the user, such that the attachment body is at least partially deformable to conform to a shape of the nose of the user, a plurality of absorption pads to absorb a fluid discharge within the nose, the plurality of absorption pads including a first of the plurality of absorption pads disposed at a first end of the attachment body, and a second of the plurality of absorption disposed at a second end of the attachment body opposite with respect to the first end, and a nasal decongestant disposed on or within at least a portion of each of the plurality of absorption pads to may alleviate or prevent a fluid discharge within the nose.

### CROSS-REFERENCE TO RELATED APPLICATIONS

This application claims priority under 35 USC § 120 from U.S. Provisional Application No. 63/536,050, entitled “Nasal Liquid Absorption Device,” which was filed on Aug. 31, 2023, in the United States Patent and Trademark Office, the disclosure of which is incorporated herein in its entirety by reference.

### BACKGROUND

### 1. Field

The present general inventive concept relates generally to a nasal insert, and particularly, to a nasal liquid absorption device.

### 2. Description of the Related Art

Rhinorrhea which is commonly known as a runny nose is a condition arising from excess discharge of mucus in a nose of a person. It often occurs due to allergies or viral infections. Although it is often not a serious condition, it can be troublesome to manage by blowing the mucus from the nose.

In general, most people will seek use of a tissue to collect the mucus blown from the nose. Also, undertaking the activity of blowing the nose in the tissue can cause an interruption such as during work or while traveling. The person may rely on the use of tissues to blow and wipe their noses, but this can become painful, causing redness and/or soreness in the nose as well as potentially increase the spread of a pathogen (e.g., a virus, bacteria, parasite, fungus).

Therefore, there is a need for a nasal liquid absorption device that absorbs any discharge from the nose without requiring use of a tissue.

### SUMMARY

The present general inventive concept provides a nasal liquid absorptiondevice.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a nasal liquid absorption device to be disposed at least partially within a nose of a user, the nasal liquid absorption device including an attachment body to removably connect to the nose of the user, such that the attachment body is at least partially deformable to conform to a shape of the nose of the user, a plurality of absorption pads to absorb a fluid discharge within the nose, the plurality of absorption pads including a first of the plurality of absorption pads disposed at a first end of the attachment body, and a second of the plurality of absorption disposed at a second end of the attachment body opposite with respect to the first end, and a nasal decongestant disposed on or within at least a portion of each of the plurality of absorption pads to may alleviate or prevent a fluid discharge within the nose.

The plurality of absorption pads may have sizes that are greater than sizes at a distance furthest from a connection point of the attachment body, such that each of the plurality of absorption pads taper from a first end to a second end thereof.

Each of the plurality of absorption pads may be at least one of wider and thicker at an opening of each nostril of the user's nose as compared to a portion deeper within the nostril.

The attachment body may be at least one of a clip, a clasp, and a clamp.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a front perspective view of a nasal liquid absorption device as disposed in a nose of a user, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the FIGURES, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Nasal Liquid Absorption Device 100
Attachment Body 110
Absorption Pads 120
Nasal Decongestant 130

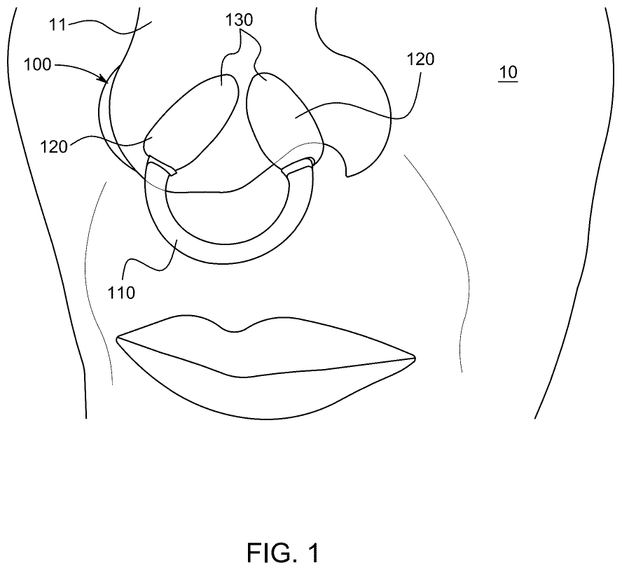

FIG. 1 illustrates a front perspective view of a nasal liquid absorption device 100 as disposed in a nose 11 of a user 10, according to an exemplary embodiment of the present general inventive concept.

The nasal liquid absorption device 100 may be constructed from at least one of metal, plastic, cloth, and rubber, etc., but is not limited thereto.

The nasal liquid absorption device 100 may include an attachment body 110, a plurality of absorption pads 120, and a nasal decongestant 130, but is not limitedthereto.

Referring to FIG. 1, the attachment body 110 is illustrated to have an elongate and cylindrical shape. However, the attachment body 110 may be rectangular, circular, triangular, pentagonal, hexagonal, heptagonal, octagonal, or any other shape known to one of ordinary skill in the art, but is not limited thereto.

The attachment body 110 may be a clip, a clasp, and a clamp, but is not limited thereto.

The attachment body 110 may be elastic. More specifically, the attachment body 110 may at least partially deform (e.g., bend, stretch, expand) in response to an application of force (e.g., pushing, pulling) thereto. Thus, the attachment body 110 may be adjust in shape based on a preference of a user 10. Additionally, the attachment body 110 may removably connect to at least a portion of a nose 11 of the user 10.

Furthermore, the attachment body 110 may remain connected to the nose 11 in absence of the application of force by clamping to the nose 11.

Each of the plurality of absorption pads 120 may include a sponge, a cloth, a cotton swap, and/or any combination thereof, but is not limited thereto.

A first of the plurality of absorption pads 120 may be disposed at a first end of the attachment body 110. Moreover, a second of the plurality of absorption pads 120 may be disposed at a second end of the attachment body 110 opposite with respect to the first end. Each of the plurality of absorption pads 120 may have a size (e.g., a diameter, a circumference) at a connection to the attachment body 110 that is greater than a size (e.g., a diameter, a circumference) at a distance furthest from the connection. In other words, each of the plurality of absorption pads 120 may taper from a first end to a second end thereof.

As such, each of the plurality of absorption pads 120 may be wider and/or thicker at an opening of each nostril compared to a portion deeper within the nostril. Therefore, each of the plurality of absorption pads 120 may be easier to insert within the nose 11 due to being narrower at a tip compared to a base where each of the plurality of absorption pads 120 is connected to the attachment body 110.

Each of the plurality of absorption pads 120 may absorb a fluid discharge within the nose 11. For example, each of the plurality of absorption pads 120 may absorb mucus, blood, water, saline solution, and/or any other type of the fluid discharge.

The nasal decongestant 130 may include phenylephrine, blood coagulant, and/or any combination thereof, but is not limited thereto.

The nasal decongestant 130 may be disposed on and/or within at least a portion of each of the plurality of absorption pads 120. The nasal decongestant 130 may alleviate and/or prevent the fluid discharge within the nose 11, such as mucus. Also, the nasal decongestant 130 may stop blood from flowing from the nose 11.

Therefore, the nasal liquid absorption device 100 may ease discomfort of cold, allergies, constant nasal drips, and/or nosebleeds. Also, the nasal liquid absorption device 100 may protect the nose 11 from irritation from use of tissues, while also keeping hands of the user 10 free.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A nasal liquid absorption device to be disposed at least partially within a nose of a user, the nasal liquid absorption device comprising:
an attachment body to removably connect to the nose of the user, such that the attachment body is at least partially deformable to conform to a shape of the nose of the user;
a plurality of absorption pads to absorb a fluid discharge within the nose, the plurality of absorption pads comprising:
a first of the plurality of absorption pads disposed at a first end of the attachment body, and
a second of the plurality of absorption disposed at a second end of the attachment body opposite with respect to the first end; and
a nasal decongestant disposed on or within at least a portion of each of the plurality of absorption pads to may alleviate or prevent a fluid discharge within the nose.

2. The nasal liquid absorption device of claim 1, wherein the plurality of absorption pads have sizes that are greater than sizes at a distance furthest from a connection point of the attachment body, such that each of the plurality of absorption pads taper from a first end to a second end thereof.

3. The nasal liquid absorption device of claim 2, wherein each of the plurality of absorption pads are at least one of wider and thicker at an opening of each nostril of the user's nose as compared to a portion deeper within the nostril.

4. The nasal liquid absorption device of claim 1, wherein the attachment body is at least one of a clip, a clasp, and a clamp.
